(19) United States
(12) Patent Application Publication
(10) Pub. No.: US 2010/0307488 A1
(43) Pub. Date: Dec. 9, 2010

(54) Tracheostomy Tube Assembly

(75) Inventors: Sylwia Ewa Krøl Poulsen, gen. Mery Valley Ipoh (MY); Mohd Akhir bin Saari, Kedah (MY)

Correspondence Address: COHEN, PONTANI, LIEBERMAN & PAVANE LLP, 551 FIFTH AVENUE, SUITE 1210, NEW YORK, NY, 10176, US

(73) Assignee: Unomedical A/S, Birkerod (DK)

(21) Appl. No.: 12/444,744

(22) PCT Filed: Oct. 16, 2006
(86) PCT No.: PCT/DK06/00579

§ 371 Date: Apr. 15, 2010

(51) Int. Cl. A61M 16/04

(52) U.S. Cl. 128/200.26

(57) ABSTRACT

The invention concerns a tracheostomy tube assembly for insertion into the trachea of a patient to support breathing, said assembly comprising an outer cannula (1) having a distal end (10) and a first connector end (11) with first connector means; an insertion member (2, 3) for insertion into the outer cannula (1) and a second connector end (21, 31) provided with second connector means, said second connector means cooperating with said first connector means for locking said insertion member (2, 3) in the outer cannula (1); wherein a swivel locking member (4) is retained on the second connector end (21, 31) of the insertion member (2, 3) so that the swivel locking member (4) extend around a portion of the first connector end (11), and said lock member (4) being provided with internal engagement means (40, 41), which is adapted to cooperate with corresponding first engagement means (42, 43) provided on the external surface (44) of the first connector end (11).

The present invention relates to tracheostomy tube assembly for insertion into the trachea of a patient to support breathing, said assembly comprising an outer cannula having a distal end and a first connector end with first connector means and an insertion member for insertion into the outer cannula and a second connector end provided with second connector means, said second connector means cooperating with said first connector means for locking said insertion member in the outer cannula.

An example of such tracheostomy tube assembly is known from WO 2004/101048 where an inner cannula is fixed in an outer cannula. Another example of such assembly of inner and outer cannulae is known from EP 0 742 729. From EP 0 824 928, an example of a tracheostomy tube assembly between the outer cannula and an obturator is shown.

Tracheostomy tubes are used to assist a patient to breathe. The tracheostomy tube is placed in a patient's throat through an opening. During the insertion of the tube, it is advantageous to insert an obturator in the outer cannula. The tip of this obturator blocks the distal end opening of the outer cannula tube and facilitates a smooth entry of the tube and prevents the ingress of tissue which could block the air passage through the tube.

When the tube is positioned, the obturator must be removed from the outer cannula so that the tube provides connection between an oxygen or air supply and the patient's trachea. When the obturator is removed, it is often that the tracheostomy tube is provided with an inner cannula, which is inserted into the outer cannula and locked to the outer cannula. The inner cannula is provided with a connector end at which point a breathing apparatus is connected to the inner cannula to facilitate the patient's breathing.

As it can be understood, it is critical that the insertion of the outer cannula, removal of the obturator and subsequent insertion and connection of the inner cannula to the outer cannula is done quickly and correctly in order to restore breathing. This means that a safe and reliable tracheostomy tube assembly is required which is easy to handle and operate quickly. This is to some extend achieved by the known tracheostomy tube assemblies. However, the known designs may be difficult to operate and in particular to disengage from each other without dislocating the outer cannula, which should be avoided since it is placed in the opening of the patient's throat when the disconnection is taking place.

Accordingly, an object of the present invention is to provide an improved tracheostomy tube assembly of the above-mentioned kind which satisfies the requirements.

This object is achieved by a tracheostomy tube assembly of the initially mentioned kind, wherein a swivel locking member is retained on the second connector end of the insertion member so that the swivel locking member extend around a portion of the first connector end, and said lock member being provided with internal engagement means, which is adapted to cooperate with corresponding first engagement means provided on the external surface of the first connector end.

By using a swivel locking mechanism, there is no relative movement between the outer cannula and the insertion member, i.e. either the obturator or the inner cannula, as the swivel lock is being operated. This facilitates both the disengagement and the locking together of the two parts and it does not require use of any force, such as a twisting force to disengage for instance a snap lock or the like. Moreover, it is found advantageous and cost-effective to use the same locking mechanism for both the obturator and the inner cannula. Thus, a major advantage of the invention is an enhanced security of the attachment between the outer cannula and the inner cannula, whereby accidental disconnection e.g. by coughing of the patient, by a nurse during the cleaning process or even patient itself is made practically impossible.

Moreover, this invention allows for a simpler design and also a cost-effective manufacturing of the outer cannula. This is particularly advantageous as the tracheostomy tubes are single use appliances and it is therefore important that the costs for manufacturing the items are kept low.

Preferably, the insertion member is provided with an annular collar for limiting the axial movement of the swivel locking member. Furthermore, the insertion member may be provided with an adaptor sleeve for retaining the swivel locking member on said second connector end of the insertion member. Hereby, the loosely fitted swivel locking member is free to rotate relative to the insertion member but retained in a correct position for performing the locking function when it is connected to the cooperating features on the outer cannula.

In a first embodiment, the first engagement means comprise at least one groove in the surface of the first connector end of the inner cannula and that the internal engagement means of the swivel locking member comprise at least one protrusion for engaging the said at least one groove and a retention collar for abutting an annular collar on the insertion member for retaining the insertion member in the outer cannula.

The grooves comprise an axially oriented groove followed by an annular groove. Alternatively, a treated portion provides said at least one groove. Through the groove the protrusion on the opposite part is inserted for locking in a bayonet locking manner.

In a second embodiment, the internal engagement means of the swivel locking member may comprise at least one groove and a retention collar for abutting an annular collar on the insertion member and said at least one groove is adapted to cooperate with the first engagement means, which comprise at least one protrusion for engaging the said at least one groove, for retaining the insertion member in the outer cannula. Similar to the variants of the first embodiment, a treated portion may provide said at least one groove or said grooves comprises an axially oriented groove followed by an annular groove.

As mentioned above, the insertion member may be an obturator as well as an inner cannula. In a tracheostomy tube kit, both an obturator and one or more inner cannulae may be provided together with an outer cannula. The advantage of the present invention is that the locking system is the same for both insertion members.

Preferably, the outer cannula is provided with a flange at said first connector end for supporting the tracheostomy tube when inserted through an opening in the throat of a patient. It is particularly preferable from a manufacturing point of view that the outer cannula is provided with an integrally moulded flange. This integrally moulded flange is connected to the outer cannula by two lateral, flexible link portions of the flange, where these link portions protect radially from the tubular portion of the outer cannula. The integral flange is provided with large areas of openings for skin oxygenation, preferably at least 50% of the circumscribed flange area.

In a preferred embodiment, the outer cannula is moulded in a plastic material with a hardness of 65-85 Shore A, and that the moulding material preferably is medical grade PVC. Moreover, the material may be compounded with a radio-opaque material, such as BaSO₄, and the material is preferably at least translucent, preferably transparent.

It is realised that the swivel locking member could alternatively be provided on the outer cannula at its connector end. However, following considerations concerning the risk of contamination and since the swivel locking member would be more likely to retain any impurities than its counter locking part, it may be found safest to provide the swivel locking nut on the part to be removed.

The invention is described in more detail with reference to a preferred embodiment shown in the drawings, in which:

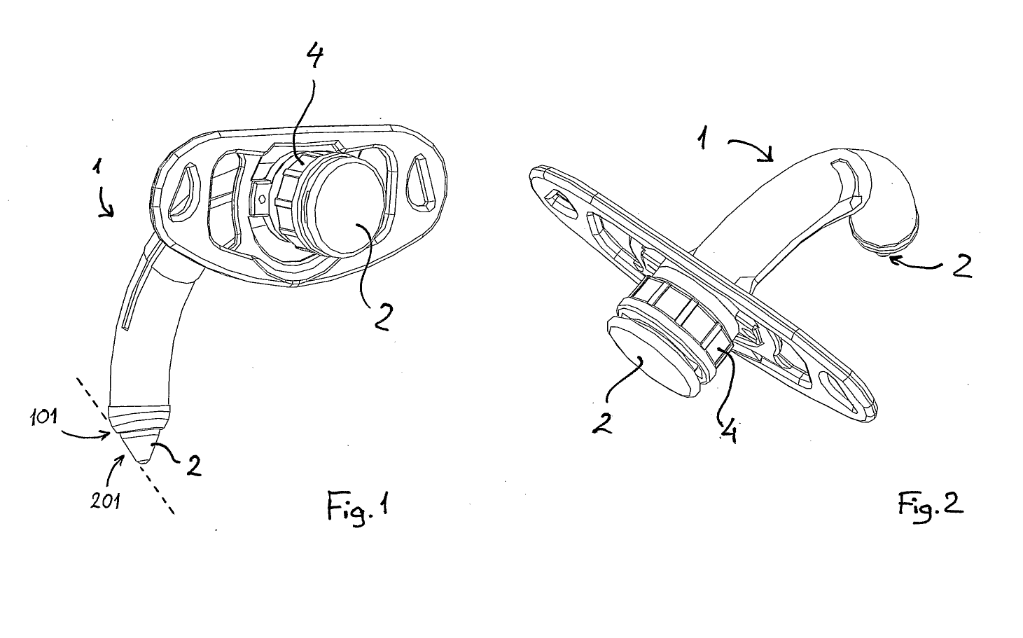

FIGS. 1 and 2 are perspective views of a tracheostomy tube assembly according to an embodiment of the invention comprising an outer cannula and an obturator;

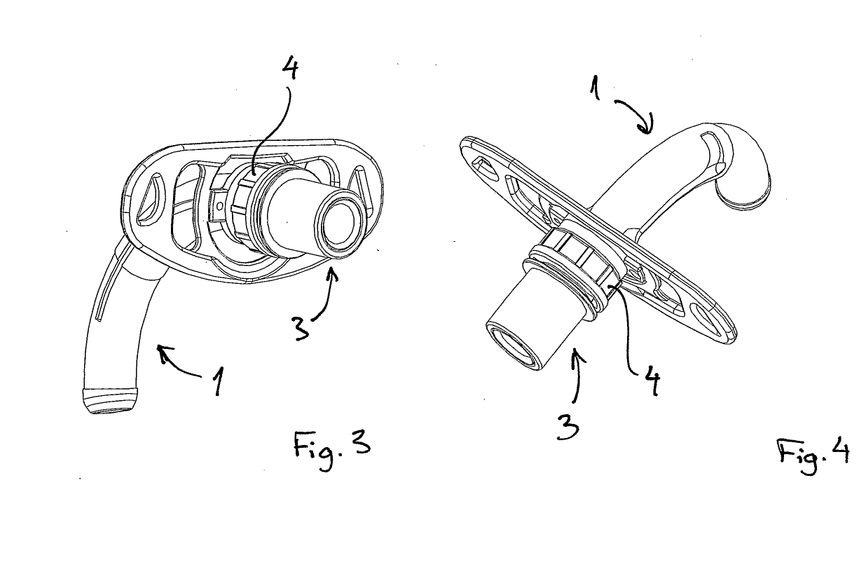

FIGS. 3 and 4 are perspective views of a tracheostomy tube assembly according to an embodiment of the invention comprising an outer cannula and an inner cannula;

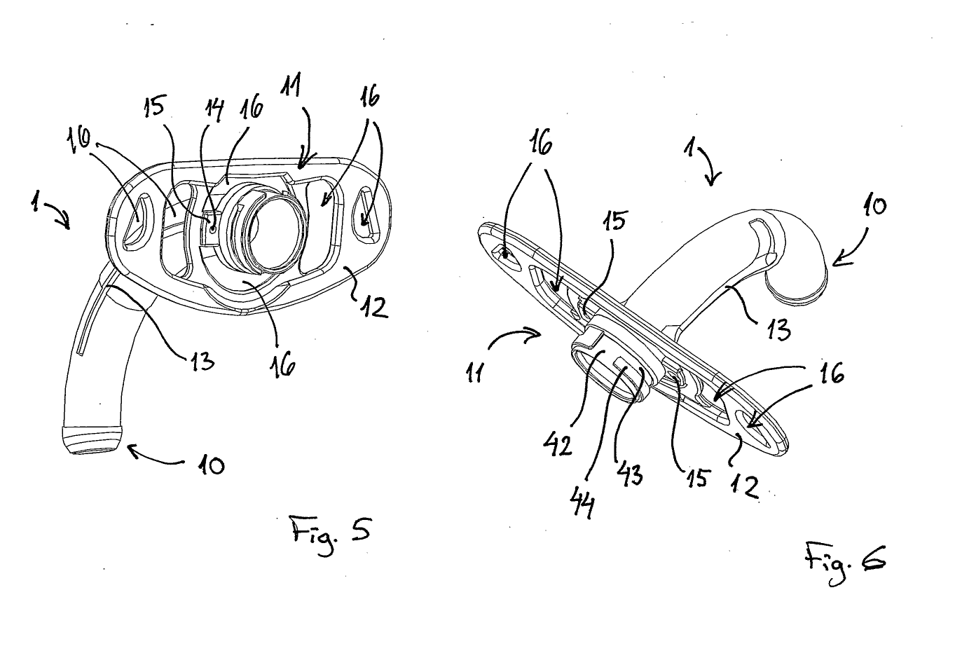

FIGS. 5 and 6 are perspective views of an outer cannula according to an embodiment of the invention;

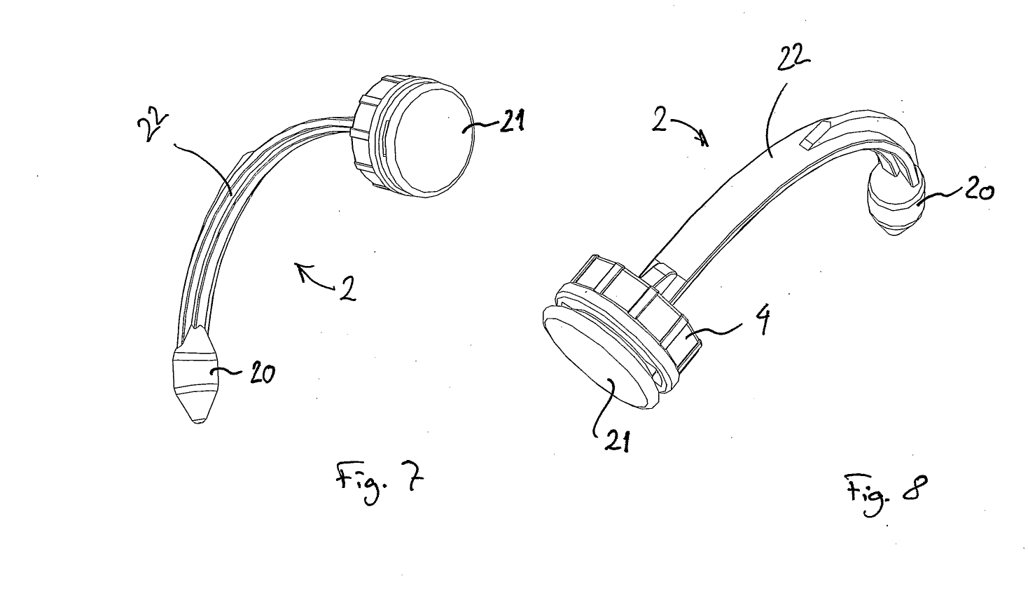

FIGS. 7 and 8 are perspective views of an obturator according to an embodiment of the invention;

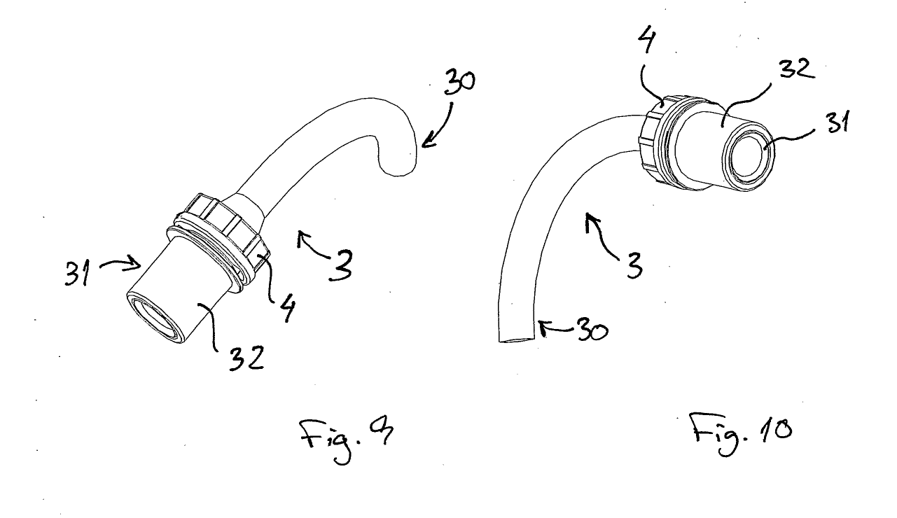

FIGS. 9 and 10 are perspective views of an inner cannula assembly according to an embodiment of the invention;

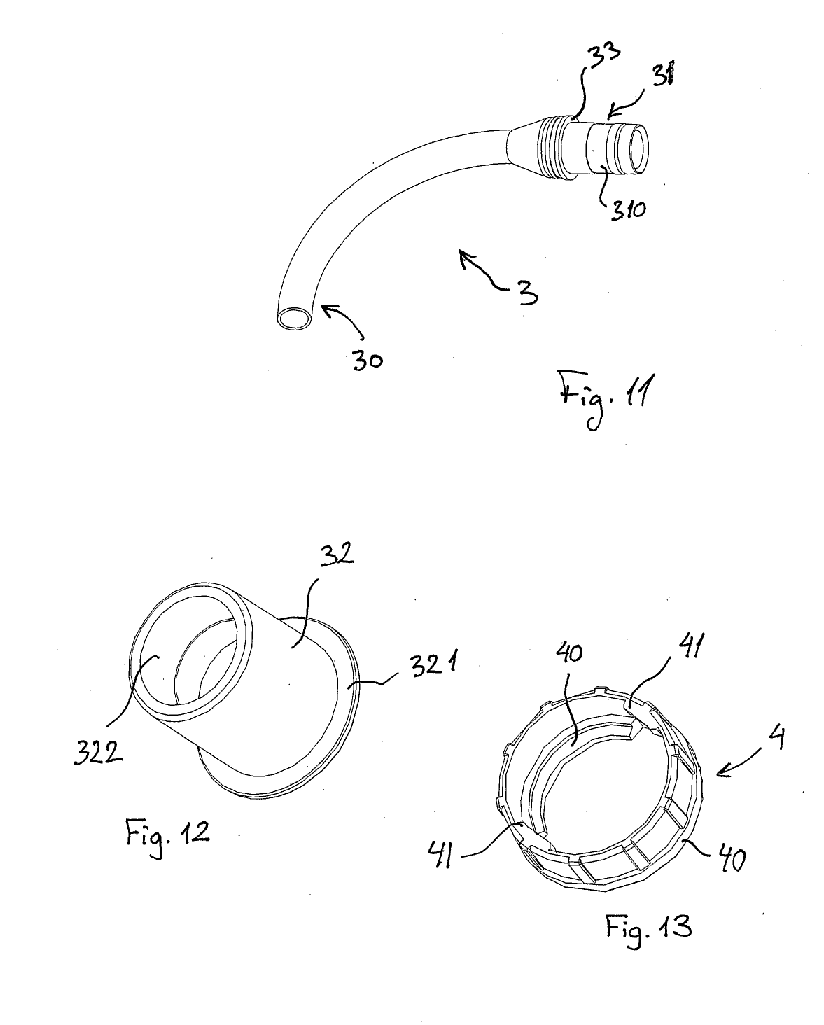

FIG. 11 is a perspective view of the inner cannula main component;

FIG. 12 is a perspective view of an end sleeve for the inner cannula according to an embodiment of the invention; and

FIG. 13 is a perspective view of a swivel locking member according to an embodiment of the invention.

In FIGS. 1 and 2 a tracheostomy tube assembly according to the invention is shown, where an outer cannula 1 is provided with an obturator 2 which is secured to the outer cannula 1 by the swivel locking member 4. In this configuration the tracheostomy tube is inserted through a puncture wound in the patient's throat and the obturator 2 ensures no dragging or scratching of the tissue during insertion and prevents tracheal wall tissue injury.

When the outer cannula 1 is positioned, the obturator 2 is removed and an inner cannula 3 is inserted into the outer cannula 1. In FIGS. 3 and 4, the tracheostomy tube assembly is shown where the inner cannula 3 is in position in the outer cannula 1. The inner cannula 3 is connected to a breathing apparatus (not shown) to facilitate the patient's breathing.

As shown in FIGS. 5 and 6, the outer cannula 1 comprises a distal end 10 and a connector end 11. At the connector end, the outer cannula 1 is provided with an integrally moulded flange 12, which is adapted to support the outer cannula 1 on the patient's skin when the tracheostomy tube assembly is in place. The flange 12 is connected to the tubular portion of the outer cannula 1 by two radially extending linking portions 15, preferably oppositely disposed. As the material the outer cannula is moulded in is a relative soft and bendable material, such as a medical grade PVC (polyvinylchloride) having a Shore A hardness of 65-90. This means that the flange 12 is flexible relative to the tubular portion of the outer cannula 1. Hereby, the flange can be rotated and angled so that movement of the flange can be absorbed in the flange 12 and not transported to the tubular portion of the outer cannula 1, whereby smaller body movements of the patient does not cause any disruptive relative movement between the tracheostomy tube, i.e. the outer cannula and the patient's body, when the tracheostomy tube is inserted through the throat of the patient.

In one of the linking portions 15 of the flange 12, an opening 14 for an inflation tube 13 is provided. The inflation tube 13 is connected to an inflatable cuff (not shown in the figures) provided near the distal end 10 of the outer cannula 1. This cuff may be inflated when the outer cannula is inserted into the trachea for stabilising it therein. In order to condition the skin around the puncture wound large openings 16 are provided in the flange 12. This not only makes the flange 12 more flexible but also oxygenises the skin during the time the tracheostomy tube is provided. By varying the flange thickness, the flange 12 may be provided with more flexibility.

The connector end 11 of the outer cannula 1 is situated on the machine end of the flange 12, i.e. the end of the outer cannula 1 at which the respirator (the breathing apparatus) is connected to the inner cannula of the tracheostomy tube. The connector end 11 is a tubular portion where the outer surface 44 is provided with grooves 42, 43, which in a preferred embodiment is provided as a first groove portion 42 which is axially oriented and open towards the extreme end of the tube portion for receiving the notch 41 of the swivel locking member 4 (see FIG. 13). This axial groove portion 42 continues in an annular groove 43, which may be essentially perpendicularly oriented relative to the first groove portion 42. The connector end 11 is preferably made of a hard material, such as ABS (Acrylonitrile butadiene styrene) and integrated into the tubular and flange portions of the outer cannula 1 by insert moulding. The hard material ensures safety of the locking device as the grip is very firm.

Turning to FIGS. 7 and 8, an obturator 2 for fitting into the outer cannula 1 (see FIGS. 5 and 6 and FIGS. 1 and 2) is shown. The obturator 2 is fitted through the machine end of the outer cannula 1 and is adapted in shape to slide through the tubular portion of the outer cannula 1 and accordingly is provided with a similar bending portion 22 of approximately 60-120 degrees, more preferably about 105° as shown in the embodiments in the figures. The obturator 2 comprises a distal end 20 formed as a outer cannula obstruction portion 20 which has a rounded smooth shape with cross sectional shape matching the tubular opening through the outer cannula 1 so it is fitted through this tubing and end by projecting the outermost portion, preferably a somewhat conical or rounded distal portion of said distal blocking portion 20 out of the distal end 10 of the outer cannula 1. The surface 201 of the distal end 20 of the obturator and the surface 101 of the distal end of the outer cannula 1 are formed so that they are aligned and create a smooth, aligned outer surface so that dragging and scratching of the tracheal wall tissue is presented during the insertion of the tracheostomy tube. The obturator 2 is provided with a connector end 21 at which a swivel locking member 4 is fitted in a manner so it is retained axially but allowed to freely rotate relative to the obturator 2.

An embodiment of the inner cannula 3 is shown in FIGS. 9 and 10. The inner cannula 3 is provided with a bed corresponding to that of the outer cannula 1 (see FIGS. 3 and 4) and it has a distal end 30. Opposite of that it has a machine end 31 where a connector sleeve 32 is fitted. The inner cannula 3 is an assembly of three components, which are individually shown in the FIGS. 11 to 13. The sleeve 32 is fitted over the distal end 31 of the inner cannula 3 as the inner surface 322 of the sleeve 32 is provided with a corresponding shape to the outer surface 310 of the distal end 31 of the inner cannula 3, so that the sleeve 32 can be axially mounted on the distal end 31 and clicked thereof so that the sleeve is axially retained on the inner cannula 3 but allowed to rotate there around. Thereby the swivel locking member 4 is retained on the inner cannula 3 and fitted loosely and axially retained by the collar 33 on the inner cannula 3 and the retaining collar 321 on the sleeve 32.

The swivel locking member 4 shown individually in FIG. 13 is adapted to be loosely fitted and allowed to rotate axially on both the obturator 2 and the inner cannula 3 for fitting the respective insertion member into the outer cannula 1 and locking it thereto in a reliable and simple manner which is easily disengageable in a manner whereby movement in the outer cannula relative to the patient may be avoided as a simple rotational movement of the swivel lock whilst holding the outer cannula is required to operate the locking and unlocking of the insertion members in a tracheostomy tube assembly according to the invention. Thus, a major advantage of the invention is the enhanced security of the attachment between the outer cannula and the inner cannula, whereby accidental disconnection e.g. by coughing of the patient, by a nurse during the cleaning process or even patient itself is made practically impossible.

1. A tracheostomy tube assembly for insertion into the trachea of a patient to support breathing, said assembly comprising:
an outer cannula having a distal end and a first connector end with first connector means;
an insertion member for insertion into the outer cannula and having a second connector end provided with second connector means, said second connector means cooperating with said first connector means for locking said insertion member in the outer cannula; and
a swivel locking member retained on the second connector end of the insertion member so that the swivel locking member extends around a portion of the first connector end, and said swivel locking member being provided with internal engagement means adapted to cooperate with corresponding first engagement means provided on the external surface of the first connector end.

2. The tracheostomy tube assembly according to claim 1, wherein said outer cannula is provided with a flange at said first connector end.

3. The tracheostomy tube assembly according to claim 1, wherein said insertion member is provided with an annular collar for limiting the axial movement of the swivel locking member.

4. The tracheostomy tube assembly according to claim 3, wherein said insertion member is provided with an adaptor sleeve for retaining the swivel locking member on said second connector end of the insertion member.

5. The tracheostomy tube assembly according to claim 1, wherein the first engagement means comprise at least one groove in the surface of the first connector end of the inner cannula and that the internal engagement means of the swivel locking member comprise at least one protrusion for engaging said at least one groove and a retention collar for abutting an annular collar on the insertion member for retaining the insertion member in the outer cannula.

6. The tracheostomy tube assembly according to claim 5, wherein said grooves comprises an axially oriented groove followed by an annular groove.

7. The tracheostomy tube assembly according to claim 5, wherein a treated portion provides said at least one groove.

8. The tracheostomy tube assembly according to claim 1, wherein the internal engagement means of the swivel locking member comprise at least one groove and a retention collar for abutting an annular collar on the insertion member and said at least one groove is adapted to cooperate with the first engagement means, which comprise at least one protrusion for engaging said at least one groove, for retaining the insertion member in the outer cannula.

9. The tracheostomy tube assembly according to claim 8, wherein a treated portion provides said at least one groove.

10. The tracheostomy tube assembly according to claim 8, wherein said grooves comprises an axially oriented groove followed by an annular groove.

11. The tracheostomy tube assembly according to claim 1, wherein said insertion member is an obturator.

12. The tracheostomy tube assembly according to claim 1, wherein said insertion member is an inner cannula.

13. The tracheostomy tube assembly according to claim 1, wherein the outer cannula is provided with an integral flange.

14. The tracheostomy tube assembly according to claim 8, wherein the integral flange is connected to the outer cannula by two lateral, flexible link portions.

15. The tracheostomy tube assembly according to claim 13, wherein the integral flange is provided with large areas of openings for skin oxygenation.

16. The tracheostomy tube assembly according to claim 1, wherein the outer cannula is moulded in a plastic material with a hardness of 65-85 Shore A.

17. The tracheostomy tube assembly according to claim 16, wherein said material is medical grade PVC.

18. The tracheostomy tube assembly according to claim 16, wherein said material is compounded with a radio-opaque material.

19. The tracheostomy tube assembly according to claim 17, wherein said material is at least translucent.

20. The tracheostomy tube assembly according to claim 19, wherein said material is transparent.
